(19) United States
(12) Patent Application Publication
(10) Pub. No.: US 2026/0347297 A1
(43) Pub. Date: Nov. 5, 2026

(54) METHOD FOR PRODUCING (POLY)ALKYLENE GLYCOL MONOALKYL ETHER

(71) Applicant: NIPPON SHOKUBAI CO., LTD., Osaka-shi, Osaka (JP)

(72) Inventors: Akiko YAMAUCHI, Kawasaki-shi, Kanagawa (JP); Hiroki KATAOKA, Kawasaki-shi, Kanagawa (JP); Toru INAOKA, Kawasaki-shi, Kanagawa (JP)

(21) Appl. No.: 19/119,814

(22) PCT Filed: Oct. 20, 2023
(86) PCT No.: PCT/JP2023/038102

§ 371 Date: Apr. 10, 2025

(30) Foreign Application Priority Data
Oct. 21, 2022 (JP) 2022-169503

(51) Int. Cl. C07C 41/06; C07C 43/11

(52) U.S. Cl. CPC C07C 41/06; C07C 43/11

(57) ABSTRACT

In a method for producing a (poly)alkylene glycol monoalkyl ether, an olefin and a (poly)alkylene glycol are reacted in the presence of a solvent, as a solution having a uniform liquid phase, using a crystalline metallosilicate as a catalyst; after the reaction, the resulting reaction solution is separated, at a temperature lower than a reaction temperature, into two phases, i.e., an upper phase mainly containing the olefin and a (poly)alkylene glycol monoalkyl ether and a lower phase mainly containing the solvent and the (poly)alkylene glycol; and then the (poly)alkylene glycol monoalkyl ether is obtained from the upper phase.

### TECHNICAL FIELD

The present invention relates to a method for producing a (poly)alkylene glycol monoalkyl ether. More particularly, the present invention relates to a method for producing a (poly)alkylene glycol monoalkyl ether by reacting an olefin with a (poly)alkylene glycol.

### BACKGROUND ART

With respect to a method for producing a (poly)alkylene glycol monoalkyl ether by reacting an olefin with a (poly)alkylene glycol, the following methods have been proposed.

JP 2703752 B (corresponding to U.S. Pat. No. 5,741,948 A) discloses that an olefin is reacted with a (poly)alkylene glycol using a BEA-type metallosilicate catalyst to obtain a (poly)alkylene glycol monoalkyl ether. It describes that the reaction can be carried out in the presence or absence of a solvent and that nitromethane, nitroethane, nitrobenzene, dioxane, ethylene glycol dimethyl ether, sulfolane, benzene, toluene, xylene, hexane, cyclohexane, decane, paraffin, or the like can be used as the solvent. WO 2021/067223 (corresponding to US 2022/0274903 A1) describes that selectivity of a monoalkyl ether is increased to 85% or more by reacting an olefin with a (poly)alkylene glycol in the presence of a solvent such as ethylene glycol dimethyl ether or diethylene glycol dimethyl ether using a BEA-type metallosilicate catalyst.

### SUMMARY OF INVENTION

However, the reaction using such a solvent is disadvantageous in terms of energy, because of a large amount of liquid to be distilled off by distillation when the reaction is carried out in a uniform solution and then the solvent, unreacted olefin, (poly)alkylene glycol, and other reaction impurities are removed to obtain a (poly)alkylene glycol monoalkyl ether, and of distillation of a reaction solution. Further, solution composition of a bottom changes during the distillation to cause phase separation, thereby causing problems of unstable distillation which causes bumping, unstable vapor composition which decreases yield of the (poly)alkylene glycol monoalkyl ether, and difficulty in controlling a degree of purification.

As a result of intensive studies to solve these problems, the present inventors have found an efficient method for producing a (poly)alkylene glycol monoalkyl ether, in which, while taking advantage of the reaction using a solvent, a reaction solution is subjected to a temperature decrease to a temperature lower than a reaction temperature after the reaction, and separated into an upper phase (A) mainly containing an olefin and a (poly)alkylene glycol monoalkyl ether and a lower phase (B) mainly containing a (poly)alkylene glycol, and thus a total amount of the upper phase (A) containing the (poly)alkylene glycol monoalkyl ether as a product is dramatically reduced, and distillation can be stably performed, whereby a highly pure product is obtained.

That is, the present invention includes the following.
(1) A method for producing a (poly)alkylene glycol monoalkyl ether, the method including:
   reacting an olefin with a (poly)alkylene glycol in the presence of a solvent, as a solution having a uniform liquid phase, at 110 to 250 °C using a crystalline metallosilicate as a catalyst;
   separating a reaction solution obtained in the reaction into an upper phase (A) and a lower phase (B) at a temperature lower than 110 °C; and
   obtaining a (poly)alkylene glycol monoalkyl ether from the upper phase (A) separated in the separation by distillation.
(2) The production method according to (1), further including removing, by distillation, a solvent component from the lower phase (B) separated in the separation, separating the lower phase (B) into an upper phase (C) and a lower phase (D), and obtaining the (poly)alkylene glycol monoalkyl ether from the separated upper phase (C) by distillation.
(3) The production method according to (1) or (2), wherein the olefin is at least one selected from the group consisting of acyclic olefins having from 6 to 30 carbon atoms.
(4) The production method according to any one of (1) to (3), wherein the crystalline metallosilicate is a pentasil-type metallosilicate or a BEA-type metallosilicate.
(5) The production method according to any one of (1) to (4), wherein the solvent used in the reaction is a compound having a solubility parameter in a range of from 16 to 20 and represented by Formula (I):

[Chem. 1]

R₁O—(AO)ₙ—R₂ (I)

where R₁ is a linear or branched alkyl group having from 1 to 4 carbon atoms, R₂ is a linear or branched alkyl group having from 1 to 4 carbon atoms or an acetyl group, A is a linear or branched alkylene group having from 2 to 4 carbon atoms, and n is from 1 to 4.
(6) A method for producing a higher secondary alcohol alkoxylate adduct, the method including further adding an alkylene oxide to the (poly)alkylene glycol monoalkyl ether produced according to any one of (1) to (5).

### DESCRIPTION OF EMBODIMENTS

Hereinafter, embodiments of the present invention will be described. It should be noted that the present invention is not limited to the following embodiments and can be variously modified within the scope of claims. The embodiments described in the present specification can be arbitrarily combined with each other to create another embodiment.

Throughout the present specification, it is to be understood that the expression of a singular form includes also a concept of a plural form thereof unless otherwise stated. Therefore, it is to be understood that an article for a singular form (for example, “a”, “an”, “the” and the like in the case of English) includes a concept of a plural form thereof, unless otherwise stated. In addition, it is to be understood that the terms used herein are used in their ordinary meanings in the art, unless otherwise stated. Accordingly, unless defined otherwise, all the technical terminologies and scientific technical terms used herein have the same meaning as commonly understood by those skilled in the art to which the present invention belongs. In case of conflict, the present specification (including definitions) is prior.

In the present specification, “from X to Y” indicating a range includes X and Y, and means “X or more and Y or less”. Unless otherwise specified, operations, measurements of physical properties, and the like are performed under conditions of room temperature (in a range of 20 °C or higher and 25 °C or lower)/relative humidity of 40% RH or more and 50% RH or less.

One aspect of the present invention relates to a method for producing a (poly)alkylene glycol monoalkyl ether, the method including: reacting an olefin with a (poly)alkylene glycol in the presence of a solvent, as a solution having a uniform liquid phase, at 110 to 250 °C using a crystalline metallosilicate as a catalyst (reaction); separating a reaction solution obtained in the reaction into an upper phase (A) and a lower phase (B) at a temperature lower than 110 °C (separation); and obtaining a (poly)alkylene glycol monoalkyl ether from the upper phase (A) separated in the separation by distillation (purification). According to the present invention, the solvent is removed in the separation, and thus the amount of the solvent to be distilled off (separated by distillation) when the reaction product is recovered can be reduced, which is preferred in terms of energy. Further, according to the present invention, an olefin and a (poly)alkylene glycol can be reacted in the presence of a catalyst with a high selectivity of a (poly)alkylene glycol monoalkyl ether, and the (poly)alkylene glycol monoalkyl ether can be produced in a high yield. According to the present invention, a (poly)alkylene glycol monoalkyl ether can be produced from an olefin and a (poly)alkylene glycol with high selectivity and in a high yield. According to the present invention, a high-purity (poly)alkylene glycol monoalkyl ether can be produced.

### [Reaction]

In this step, an olefin is reacted with a (poly)alkylene glycol at a temperature of from 110 to 250 °C in the presence of a crystalline metallosilicate (catalyst) using a solvent which provides a solution having a uniform liquid phase. Typically, the raw material olefin is not miscible with a (poly)alkylene glycol (for example, ethylene glycol), and thus is easily separated into two phases after the reaction. Therefore, an olefin phase containing the reaction product ((poly)alkylene glycol monoalkyl ether) is easily separated, and the (poly)alkylene glycol and the catalyst can be recovered. However, the reaction needs to be carried out under vigorous stirring because of easy separation. In addition, as described above, it is known to use a solvent in the reaction for promoting the reaction, but the solvent needs to be distilled off (separated by distillation) to recover the reaction product, which is disadvantageous in terms of energy. On the other hand, according to the present invention, the reaction can be carried out in a uniform solution state, and thus the reaction can be carried out without requiring vigorous stirring. When the temperature is lowered, phase separation is easily performed into an upper phase mainly containing the reaction product ((poly)alkylene glycol monoalkyl ether) and the unreacted olefin and the solvent and a lower phase mainly containing the solvent and the unreacted (poly)alkylene glycol. Therefore, the reaction product can be recovered easily and reliably (in a high yield/high yield).

In the present specification, “solution having a uniform liquid phase” means that the liquid phase is a single phase. Specifically, whether or not a solution is a solution having a uniform liquid phase is evaluated based on whether or not the liquid phase is visually separated into two phases. When the liquid phase is not separated into two phases and is a single phase, the solution is evaluated as a solution having a uniform liquid phase.

The olefin used in the present invention is, for example, a hydrocarbon having an ethylenically unsaturated bond and from 2 to 40 carbon atoms, preferably a hydrocarbon having an ethylenically unsaturated bond and from 6 to 30 carbon atoms, more preferably a hydrocarbon having one ethylenically unsaturated bond and from 8 to 20 carbon atoms, and particularly preferably an acyclic hydrocarbon having one ethylenically unsaturated bond and from 12 to 16 carbon atoms (acyclic olefin) (dodecene, tridecene, tetradecene, pentadecene, and hexadecene). Specific examples of the olefin include ethylene, propylene, butene, isobutylene, butadiene, pentene, hexene, octene, nonene, decene, undecene, dodecene, tridecene, tetradecene, pentadecene, hexadecene, heptadecene, octadecene, nonadecene, eicosene, heneicosene, docosene, tricosene, and tetracosene. These may be used alone, or two or more kinds thereof may be used as a mixture. In particular, olefins having a large number of carbon atoms, such as octene, decene, dodecene, tetradecene, hexadecene, octadecene, and eicosene, are suitably used. These olefins can be used without particular limitation regardless of whether the position of the unsaturated bond is α-position, inner-position, or both α-position and inner-position. In an embodiment of the present invention, the olefin has an unsaturated bond at α-position (e.g., 1-dodecene, 1-tridecene, 1-tetradecene). In an embodiment of the present invention, the olefin has an unsaturated bond at inner-position (e.g., an inner-dodecene, an inner-tridecene, an inner-tetradecene). Of course, two or more kinds of these olefins different in unsaturated bond position may be used in combination. In an embodiment of the present invention, the olefin is a mixture of an olefin in which the unsaturated bond position is α-position and an olefin in which the unsaturated bond position is inner-position (e.g., a mixture of 1-dodecene and an inner-dodecene, a mixture of 1-tridecene and an inner-tridecene, a mixture of 1-tetradecene and an inner-tetradecene). In the reaction process of the present invention, an isomerization reaction of the olefin for the unsaturated bond position occurs simultaneously. In general, an inner-olefin is thermodynamically more stable than an α-olefin, and thus, when an α-olefin is used as a raw material, the α-olefin is gradually isomerized into an inner-olefin during the reaction. A rate of isomerization varies depending on the reaction temperature and the type and amount of the crystalline metallosilicate used as the catalyst. The (poly)alkylene glycol monoalkyl ether obtained from the acyclic olefin having from 6 to 30 carbon atoms is suitable as a raw material for a surfactant.

Examples of the (poly)alkylene glycol used in the present invention include monoethylene glycol, diethylene glycol, triethylene glycol, polyethylene glycol, monopropylene glycol, dipropylene glycol, tripropylene glycol, polypropylene glycol, 1,3-propanediol, 1,2-butanediol, 2,3-butanediol, 1,4-butanediol, 1,6-hexanediol, paraxylylene glycol, and 1,4-cyclohexanemethanediol. These may be used alone, or two or more kinds thereof may be used as a mixture. Among these, monoethylene glycol, diethylene glycol, triethylene glycol, and monopropylene glycol are preferred, and monoethylene glycol is more preferred. According to an embodiment of the present invention, the alkylene in the (poly)alkylene glycol has from 1 to 8, from 1 to 6, from 1 to 4, or from 1 to 3 carbon atoms. According to an embodiment of the present invention, the number of moles of alkylene oxide added to the (poly)alkylene glycol can be from 1 to 20.

The crystalline metallosilicate used in the present invention is an ordered porous material having a certain crystal structure. That is, it is a solid substance having a large number of ordered voids or holes in its structure and a large specific surface area.

The crystalline metallosilicate used in the present invention is a crystalline aluminosilicate (generally also referred to as zeolite) or a compound in which another metal element is introduced into the crystal lattice instead of the Al atom of the crystalline aluminosilicate. Specific examples of such other metal atoms include at least one of B, Ga, In, Ge, Sn, P, As, Sb, Sc, Y, La, Ti, Zr, V, Cr, Mn, Fe, Co, Ni, Cu, and Zn. In terms of catalytic activity and ease of synthesis and availability, crystalline aluminosilicate, crystalline ferrosilicate, crystalline borosilicate, and crystalline gallosilicates are preferred, and crystalline aluminosilicate is more preferred.

According to an embodiment of the present invention, the catalyst has a specific surface area of from 400 to 800 m²/g.

Specific examples of the crystalline metallosilicate used in the present invention include those having structures such as MFI (ZSM-5 and the like), MEL (ZSM-11 and the like), BEA (B-type zeolite and the like), FAU (Y-type zeolite and the like), MOR (mordenite and the like), MTW (ZSM-12 and the like), and LTL (L-type zeolite and the like) when described using IUPAC codes.

In addition to these, those having structures described in “ZEOLITES, Vol. 12, No. 5, 1992” and “HANDBOOK OF MOLECULAR SIEVES, written by R. Szostak, published by VAN NOSTRAND REINHOLD” can be exemplified. These may be used alone, or two or more kinds thereof may be used in combination. Among them, those having a structure such as MFI or MEL referred to as a pentasil type or a structure of BEA are preferred because of their excellent catalytic activity. That is, in a preferred embodiment of the present invention, the crystalline metallosilicate is a pentasil-type metallosilicate or a BEA-type metallosilicate.

The crystalline metallosilicate used in the present invention preferably has an atomic ratio of silicon atoms to metal atoms constituting it of 5 or more and 1500 or less, and particularly 10 or more and 500 or less. Those having an excessively small or large atomic ratio of the silicon atoms to the metal atoms are not preferred because of their low catalytic activity.

Many of these crystalline metallosilicates have ion-exchangeable cations outside the crystal lattice. Specific examples of these cations include H⁺, Li⁺, Na⁺, Rb⁺, Cs⁺, Mg²⁺, Ca²⁺, Sr²⁺, Ba²⁺, Sc³⁺, Y³⁺, La³⁺, R₄N⁺, and R₄P⁺, where R is H or an alkyl group. Among them, those in which all or some of cations are substituted with hydrogen ions (H⁺) are suitable as the catalyst of the present invention.

The crystalline metallosilicate used in the present invention can be synthesized by a generally used synthesis method, for example, a hydrothermal synthesis method. Specifically, it can be synthesized by a method described in JP 46-10064 B, U.S. Pat. No. 3,965,207, “Journal of Molecular Catalysis”, Vol. 31, pp. 355 to 370 (1985), and the like. The crystalline metallosilicate can be synthesized, for example, by heating a composition containing a silica source, a metallo source, and a quaternary ammonium salt such as a tetrapropylammonium salt at a temperature of about from 100 to 175 °C until crystals are formed, then filtering a solid product, washing it with water, drying it, and firing it at 350 to 600 °C. As the silica source, water glass, silica sol, silica gel, alkoxysilane, and the like can be used. As the metallo source, various inorganic or organic metal compounds can be used. Suitable examples of the metal compounds include metal salts such as metal sulfates [for example, Al₂(SO₄)₃], metal nitrates [for example, Fe(NO₂)₂], and alkali metal salts of metal oxides [for example, NaAlO₂]; metal halides such as metal chlorides [for example, TiCl₄] and metal bromides [for example, MgBr₂]; and metal alkoxides [for example, Ti(OC₂H₅)₄]. The obtained crystalline metallosilicate can be ion-exchanged into a cation form of interest, if necessary. For example, the H⁺-type cation form can be prepared by mixing and stirring the crystalline metallosilicate in an aqueous solution of HCl, NH₄Cl, NH₃, or the like to exchange the cationic species to the H⁺-type or NH₄⁺-type, then filtering a solid product, washing it with water, drying it, and firing it at 350 to 600 °C. A cation form other than H⁺ can be prepared by performing the same procedure using an aqueous solution containing the cation of interest.

Alternatively, the crystalline metallosilicate used in the present invention may be a commercially available product. Examples of the commercially available products include HSZ-900 series (BEA), HSZ-800 series (ZSM-5), HSZ-600 series (mordenite), HSZ-500 series (L-type), and HSZ-300 series (Y-type) zeolites (all available from Tosoh Corporation), CP series, CBV series, and ZD series (all available from Zeolyst International), CZB series, CZP series, CZM series, CZC series, LiCZF series, CZA series, CZE series, and CZL series (all available from Clariant AG).

The crystalline metallosilicate may be fired prior to the reaction. Here, firing conditions are not particularly limited and can be appropriately selected depending on the desired degree of catalytic activity and the like. A firing temperature is, for example, from 300 to 1000 °C, and particularly from 500 to 700 °C. The firing time is, for example, from 1 to 10 hours, and particularly from 1 to 5 hours. The firing may also be performed in air or under an inert atmosphere (e.g., nitrogen and argon).

The catalyst used in this reaction may be in any form, such as a powder form, a granular form, or a molded body having a specific shape. When a molded body is used, alumina, silica, titania, or the like may be used as a carrier or a binder.

In the present invention, an olefin is reacted with a (poly)alkylene glycol in the presence of a crystalline metallosilicate (catalyst) using a solvent which provides a solution having a uniform liquid phase. Here, the solvent that can be used in the reaction is a solvent which provides a solution having a uniform liquid phase (enables a uniform reaction). The solvent that can be used in the reaction preferably contains a compound having a solubility parameter of from 16 to 20 and represented by the following Formula (I). More preferably, the solvent that can be used in the reaction is only a compound having a solubility parameter of from 16 to 20 and represented by the following Formula (I).

That is, in the present invention, the reaction between the olefin and the (poly)alkylene glycol is preferably performed in the presence of a compound having a solubility parameter in a range of from 16 to 20 and represented by the following Formula (I). In a preferred embodiment of the present invention, the solvent used in the reaction includes a compound having a solubility parameter in the range of from 16 to 20 and represented by the following Formula (I). In a more preferred embodiment of the present invention, the solvent used in the reaction is a compound having a solubility parameter in the range of from 16 to 20 and represented by the following Formula (I). In a particularly preferred embodiment of the present invention, the solvent used in the reaction is one kind of compound having a solubility parameter in the range of from 16 to 20 and represented by the following Formula (I).

[Chem. 2]

R₁O—(AO)ₙ—R₂ (I)

In the Formula (I) above, R₁ is a linear or branched alkyl group having from 1 to 4 carbon atoms. Examples of the alkyl group as R₁ include a methyl group, an ethyl group, a propyl group, an isopropyl group, a butyl group, a sec-butyl group, and a tert-butyl group. Among these, R₁ is preferably a methyl group, an ethyl group, or a butyl group, more preferably a methyl group or an ethyl group, and particularly preferably a methyl group, from the viewpoint of further uniformity of the liquid phase during the reaction. R₂ is a linear or branched alkyl group having from 1 to 4 carbon atoms, or an acetyl group (CH₃CO—). Here, specific examples of the alkyl group as R₂ are the same as those of R₁ described above. Among these, R₂ is preferably a methyl group, an ethyl group, a butyl group, or an acetyl group, more preferably a methyl group or an ethyl group, and particularly preferably a methyl group, from the viewpoint of further uniformity of the liquid phase during the reaction. A is a linear or branched alkylene group having from 2 to 4 carbon atoms. When a plurality of As are present (n is from 2 to 4), the As may be the same or different. Examples of the alkylene group as A include an ethylene group (—CH₂CH₂—), a trimethylene group (—CH₂CH₂CH₂—), a propylene group (—CH(CH₃) CH₂— or —CH₂CH(CH₃)—), and a tetramethylene group (—CH₂CH₂CH₂CH₂—). Among these, A is preferably an ethylene group or a propylene group, and particularly preferably an ethylene group, from the viewpoint of further uniformity of the liquid phase during the reaction. n is from 1 to 4. From the viewpoint of further uniformity of the liquid phase during the reaction, m is more preferably from 1 to 3, further preferably 1 or 2, and particularly preferably 2.

The solubility parameter (SP value) of the solvent is from 16 to 20. From the viewpoint of further uniformity of the liquid phase during the reaction, and the like, the solubility parameter is preferably from 16.5 to 19.0, more preferably 17.0 or more and less than 19.0, and particularly preferably from 17.5 to 18.5.

In the present invention, the solubility parameter (SP value) refers to a Hansen solubility parameter, and is an index serving as a measure of the solubility of a two-component solution.

As a calculation method of the SP value δ (J/cm³) of the solvent, the following equation (II) is used.

[Math. 1]

δ=((δ_{d}²+δₚ²+δₕ²))^{1/2} (II)

In the equation (II), δ_{d} is a London dispersion force term, δₚ is a molecular polarization term, and δₕ is a hydrogen bond term.

Further, the solubility parameter can be calculated by Hansen solubility parameter software (HSPiP ver 5.3.08). A mixture of a plurality of these solvents may be used.

As a specific solvent, examples of a dialkyl glycol ether in which R₁ and R₂ in the Formula (I) above are alkyl groups include ethylene glycol dimethyl ether (SP value=17.9), diethylene glycol dimethyl ether (SP value=18.3), triethylene glycol dimethyl ether (SP value=18.4), ethylene glycol diethyl ether (SP value=17.1), diethylene glycol diethyl ether (SP value=17.5), diethylene glycol dibutyl ether (SP value=17.0), propylene glycol dimethyl ether (SP value=17.0), and propylene glycol diethyl ether (SP value=16.6). Examples of a glycol ether acetate in which R₁ is an alkyl group and R₂ is an acetyl group include ethylene glycol monomethyl ether acetate (SP value=19.3), ethylene glycol monoethyl ether acetate (SP value=18.6), ethylene glycol monobutyl ether acetate (SP value=18.0), diethylene glycol monoethyl ether acetate (SP value=18.6), diethylene glycol monobutyl ether acetate (SP value=18.1), propylene glycol monomethyl ether acetate (SP value=18.3), propylene glycol monoethyl ether acetate (SP value=17.8), and dipropylene glycol monomethyl ether acetate (SP value=18.3). Particularly preferred solvents include ethylene glycol dimethyl ether and diethylene glycol dimethyl ether. Each of the solvents described above may be used alone or in the form of a mixture of two or more kinds thereof, but is preferably used alone.

The reaction in the present invention is characterized in that an olefin and a (poly)alkylene glycol are reacted in the presence of a solvent, as a solution having a uniform liquid phase, at 110 to 250 °C using a crystalline metallosilicate as a catalyst. When a solvent is not used, the raw materials (poly)alkylene glycol and olefin are distributed to each other only in amounts corresponding to a slight solubility, the catalyst crystalline metallosilicate is distributed to a (poly)alkylene glycol phase, and the product (poly)alkylene glycol monoalkyl ether is distributed to an olefin phase, whereby the reaction rate becomes very slow. However, the liquid phase becomes uniform due to the use of the solvent, the olefin and the catalyst easily come into contact with each other, and thus the rate of the reaction with the (poly)alkylene glycol is increased. Here, “the liquid phase becomes uniform” means that the liquid phase becomes uniform at a reaction temperature of 110 °C or higher, the liquid phase becomes uniform when the temperature is raised or the reaction proceeds (for example, 120 minutes after a predetermined reaction temperature is reached), and the liquid phase does not have to become uniform when mixing is performed at a temperature lower than 110 °C. An amount of the solvent to be used, which makes the liquid phase uniform as described above, is preferably 35 mass % or more, more preferably 60 mass % or more, further preferably 80 mass % or more, and particularly preferably more than 100 mass % with respect to a total amount of the olefin, the (poly)alkylene glycol, and the solvent. An upper limit of the amount of the solvent to be used is, for example, 300 mass % or less, preferably 250 mass % or less, and more preferably 200 mass % or less with respect to the total amount of the olefin, the (poly)alkylene glycol, and the solvent.

Alternatively or additionally, an amount of solvent to be added is more than 62 parts by mass with respect to 100 parts by mass of the olefin. Below the lower limit, the liquid phase during the reaction is separated into two phases and does not become uniform. From the viewpoints of further improvement in uniformity of the liquid phase (for example, uniformity of the liquid phase from the start of the reaction), further ease of separation in the separation, and the like, the solvent is supplied to the reaction in a proportion of preferably from 150 to 800 parts by mass, more preferably from 165 to 600 parts by mass, further preferably more than 165 parts by mass and 500 parts by mass or less, and particularly preferably from 250 to 350 parts by mass with respect to 100 parts by mass of the olefin.

The reaction in the present invention can be performed by a generally used method such as a batch reaction or a flow reaction, and is not particularly limited. A molar ratio between the olefin and the (poly)alkylene glycol, which are raw materials for the reaction, is not particularly limited. The (poly)alkylene glycol is used in a proportion of, for example, from 0.05 to 20 mol, preferably from 0.1 to 10 mol, and more preferably from 1 to 5 mol with respect to 1 mol of the olefin.

The reaction temperature is from 110 to 250 °C, preferably from 110 to 200 °C, more preferably from 110 to 170 °C, and particularly preferably from 120 to 150 °C. A high reaction temperature is not preferred, because the reaction rate is high, but byproducts are generated by side reactions. A reaction pressure may be reduced pressure, normal pressure or increased pressure, but is preferably in a range of from normal pressure to 2 MPa.

When a batch reactor is used, the catalyst and raw materials of the present invention are filled into a reactor and stirred at a predetermined temperature and a predetermined pressure to obtain a mixture containing the (poly)alkylene glycol monoalkyl ether of interest. An amount of the crystalline metallosilicate (catalyst) to be used (added) is not particularly limited and is appropriately selected depending on the desired extent of reaction and the like.

The amount of the crystalline metallosilicate (catalyst) is from 0.1 to 150 mass %, preferably from 0.5 to 100 mass %, more preferably from 10 to 100 mass %, and particularly preferably from 20.0 to 99.5 mass %, with respect to the olefin as a raw material.

A reaction time varies depending on the reaction temperature, the amount of catalyst, the raw material composition ratio, and the like, and is in a range of from 0.1 to 100 hours, preferably from 0.5 to 30 hours, preferably from 1 to 10 hours, and more preferably from 2 to 5 hours.

[Separation]

In this step, the reaction solution obtained in the reaction is separated into an upper phase (A) and a lower phase (B) at a temperature lower than 110 °C.

In the separation of the present invention, in order to obtain a (poly)alkylene glycol monoalkyl ether from a reaction solution obtained in the reaction, the reaction solution is allowed to stand at a temperature lower than 110 °C to separate the reaction solution into two phases: an upper phase (A) mainly containing the solvent, an unreacted olefin and the product (poly)alkylene glycol monoalkyl ether; and a lower phase (B) mainly containing the solvent and an unreacted (poly)alkylene glycol. A temperature for separation (separation temperature) is lower than 110 °C, and is preferably 100 °C or lower, more preferably 90 °C or lower, further preferably 50 °C or lower, and particularly preferably lower than 40 °C. The temperature for separation (separation temperature) is a temperature at which freezing does not occur, and is, for example, −20 °C or higher, preferably 10 °C or higher, and more preferably 20 °C or higher. Alternatively, from the viewpoint of further ease of separation between the upper phase (A) and the lower phase (B), a difference between the reaction temperature in the reaction and the separation temperature in this step is, for example, from 10 to 200 °C, preferably from 30 to 150 °C, more preferably from 50 to 140 °C, further preferably from 70 to 130 °C, and particularly preferably higher than 95 °C and lower than 120 °C. Further, the separation may be performed after removal of the catalyst by filtration or centrifugal separation, or may be performed while the catalyst is contained. The catalyst separated by the method such as centrifugal separation or filtration can be used for the repeated reaction, as it is or after a regeneration treatment.

An amount of the solvent suitable for providing a uniform liquid phase during the reaction and separating the reaction solution into two phases after the reaction is preferably 35 mass % or more, more preferably 38 mass % or more, and further preferably 40 mass % or more, with respect to the total amount of the olefin, the (poly)alkylene glycol, and the solvent. An amount of the solvent of less than 35 mass % is not preferred, because the liquid phase of the reaction solution is not uniform at the reaction temperature, and the reaction rate becomes slow. An amount of the solvent of more than 65 mass % is not preferred for the following reasons. The liquid phase of the reaction solution becomes uniform, but the reaction solution is not separated even when the temperature of the reaction solution is lower than 110 °C, and the amount of the reaction solution increases to obtain a (poly)alkylene glycol monoalkyl ether. In addition, a large amount of distillation energy is required when the unreacted olefin, the unreacted (poly)ethylene glycol, the solvent, and the like are recovered and reused for the reaction. The amount is preferably 65 mass % or less, more preferably 60 mass % or less, and further preferably 55 mass % or less.

As a method for adjusting the amount of the solvent suitable for the separation, there is a method for adjusting the amount of the solvent at the time of charging the reaction or a method for adjusting the amount of the solvent after the reaction by distillation or the like. When the solvent is used in an amount larger than the amount thereof suitable for the separation, the solvent can be removed from the reaction solution by distillation to adjust the amount of the solvent so that the amount of the solvent becomes suitable for the separation after the reaction. That is, the present invention includes a method in which the reaction is carried out by adjusting the amount of the solvent to an amount suitable for the separation in the reaction and/or a method in which the solvent is removed from the reaction solution by distillation after the reaction to adjust the amount of the solvent to an amount suitable for the separation. Distillation conditions are not particularly limited, and a known method can be used. As a distillation temperature, for example, a column top temperature of a distillation column is usually from 50 to 300 °C. The distillation may be carried out under normal pressure or reduced pressure, preferably under reduced pressure, and a degree of pressure reduction is preferably 15 kPa or less, more preferably 10 kPa or less. The degree of pressure reduction is preferably 50 Pa or more, more preferably 100 Pa or more.

### [Purification]

In this step, a (poly)alkylene glycol monoalkyl ether is obtained by distillation from the upper phase (A) separated in the separation described above.

The step of obtaining a (poly)alkylene glycol monoalkyl ether by distillation from the upper phase (A) separated in the separation of the present invention is a step of obtaining a (poly)alkylene glycol monoalkyl ether by distilling off low-boiling point substances such as the solvent and the unreacted olefin because the upper phase (A) mainly contains the solvent, the unreacted olefin, and the product (poly)alkylene glycol monoalkyl ether. Distillation conditions are not particularly limited, and a known method, for example, the same method as that for the distillation of the solvent described above can be used. The obtained (poly)alkylene glycol monoalkyl ether can be used as a raw material for a higher secondary alcohol alkoxylate adduct which will be described later, as it is or after purification.

The lower phase (B) contains a part of the (poly)alkylene glycol monoalkyl ether of interest and the unreacted olefin in addition to the catalyst, the unreacted (poly)alkylene glycol, and the solvent. The lower phase (B) can also be reused for the reaction for the (poly)alkylene glycol monoalkyl ether, by addition of the olefin and/or (poly)alkylene glycol as raw material(s) as necessary. Alternatively, by removing a solvent component from the lower phase (B) by distillation, the lower phase (B) can be separated into an upper phase (C) mainly containing the unreacted olefin and the (poly)alkylene glycol monoalkyl ether as a reaction product and a lower phase (D) mainly containing the unreacted (poly)alkylene glycol. That is, the present invention may further include removing, by distillation, the solvent from the lower phase (B), separating the lower phase (B) into an upper phase (C) and a lower phase (D), and obtaining the (poly)alkylene glycol monoalkyl ether from the separated upper phase (C) by distillation. Thus, the (poly)alkylene glycol monoalkyl ether can be obtained also from the lower phase (B), and the (poly)alkylene glycol monoalkyl ether produced by the reaction can be recovered without waste. When the solvent component is removed from the lower phase (B) by distillation, the removal may be performed after the catalyst is removed by filtration or centrifugal separation, or may be performed while the catalyst is contained. The catalyst separated by the method such as centrifugal separation or filtration can be used for the repeated reaction, as it is or after a regeneration treatment.

In an embodiment of the present invention, the method of the present invention further includes removing, by distillation, a solvent component from the lower phase (B) separated in the separation, separating the lower phase (B) into an upper phase (C) and a lower phase (D), and obtaining the (poly)alkylene glycol monoalkyl ether from the separated upper phase (C) by distillation.

The (poly)alkylene glycol monoalkyl ether is obtained by distillation from the separated upper phase (A) and upper phase (C), but the unreacted olefin and solvent can also be recovered by distillation and reused as the reaction raw material and solvent. The unreacted (poly)alkylene glycol is contained also in the lower phase (D), and can be reused as a reaction raw material, as it is or after purification.

Distillation conditions are not particularly limited, and a known method can be used. As a distillation temperature, for example, the column top temperature of a distillation column is usually from 50 to 300 °C, preferably from 70 to 300 °C, and more preferably from 70 to 250 °C. A distillation residence time is usually 24 hours or less, preferably 12 hours or less, and more preferably 6 hours or less. The distillation residence time is preferably 10 minutes or more, more preferably 20 minutes or more, and further preferably minutes or more. The distillation may be carried out under normal pressure or reduced pressure, preferably under reduced pressure, and a degree of pressure reduction is preferably 15 kPa or less, more preferably 10 kPa or less. The degree of pressure reduction is preferably 50 Pa or more, more preferably 100 Pa or more.

When a flow reactor is used, the reaction can be carried out by any of a fluidized bed type, a fixed bed type and a stirred tank type. Reaction conditions vary depending on the raw material composition, catalyst concentration, reaction temperature, and the like, but a liquid hourly space velocity (LHSV), that is, a value obtained by dividing a volume flow rate of a flowing raw material by a volume of the reactor, is in a range of from 0.01 to 50 hr⁻¹, particularly from 0.1 to 20 hr⁻¹. After completion of the reaction, the (poly)alkylene glycol monoalkyl ether of interest can be recovered by the same procedure as in the batch reaction.

### [Method for Producing Higher Secondary Alcohol Alkoxylate Adduct]

The present invention provides a method for producing a higher secondary alcohol alkoxylate adduct, the method including further adding an alkylene oxide to the (poly)alkylene glycol monoalkyl ether obtained above in the presence of a catalyst.

The method for producing a higher secondary alcohol alkoxylate adduct of the present invention is not particularly limited as long as it is a method capable of adding an alkylene oxide to the (poly)alkylene glycol monoalkyl ether, and a known method can be applied in the same manner or with appropriate modification. For example, an alkylene oxide is added to the (poly)alkylene glycol monoalkyl ether obtained by the above-described method to attain an appropriate molar ratio, and the mixture is reacted in the presence of an alkali catalyst, an acid catalyst, a solid acid catalyst such as alumina magnesia, a double metal cyanide (DMC) catalyst, or the like. One kind of the alkylene oxide may be used alone, or two or more kinds of the alkylene oxides may be used in combination. In the latter case, two or more kinds of the alkylene oxides may be added randomly, each may be added in a block form, or a random structure and a block structure may be combined.

Examples of the alkylene oxide include ethylene oxide, propylene oxide, butylene oxide, and styrene oxide. A molar ratio of the alkylene oxide to the (poly)alkylene glycol monoalkyl ether is not particularly limited, and is preferably from 1 to 30, more preferably from 2 to 25, and more preferably from 4 to 20, from 3 to 18, from 4 to 16, or from 5 to 12.

As reaction conditions for adding the alkylene oxide to the (poly)alkylene glycol monoalkyl ether, a reaction temperature is usually from 50 to 250 °C, preferably from 100 to 200 °C, from 110 to 180 °C, or from 120 to 160 °C. According to an embodiment of the present invention, a reaction time is from 0.5 to 40 hours, from 0.5 to 20 hours, or from 1 to 10 hours. The reaction pressure may be normal pressure or increased pressure, and is preferably in a range of from normal pressure to 2 MPa (1 MPa or less, or 0.5 MPa or less). A reaction temperature of lower than 50 °C is not preferred because a reaction rate may become slow, and a reaction temperature of higher than 250 °C is not preferred because decomposition and an increase in byproducts may occur.

Examples of the alkali catalyst include hydroxides of elements belonging to alkali metals or alkaline earth metals, such as sodium hydroxide, potassium hydroxide, lithium hydroxide, magnesium hydroxide, calcium hydroxide, and barium hydroxide. In view of availability and reactivity, sodium hydroxide, potassium hydroxide, and the like are preferred. These may be added in the form of a powder, granules, or, further, an aqueous solution, followed by dehydration. An amount of the alkali catalyst to be used (in terms of solid content when in the form of an aqueous solution) is from 0.01 to 2.0 mass %, preferably from 0.02 to 0.5 mass %, with respect to the (poly)alkylene glycol monoalkyl ether as a raw material.

### EXAMPLES

Hereinafter, the present invention will be described in more detail with reference to examples. However, the examples are aspects of the present invention, and the present invention is not limited thereto. Unless otherwise specified, the procedure is performed at room temperature (from 20 to 25 °C).

### (Preparation of Catalyst (1))

BEA-type zeolite available from Tosoh Corporation (trade name: HSZ-930NHA, powder product, pore size: 6.5 Å, cation: template, SiO₂/Al₂O₃ ratio:27 (molar ratio), specific surface area: 590 m²/g (BET method), crystal size: 0.04 μm, particle size: 5 μm) was fired in air at 500 °C for 4 hours to obtain a catalyst (1).

### (Calculation of Conversion, Selectivity, and Yield)

From the analytical values of the phases obtained in Examples and Comparative Examples by gas chromatography (GC), the conversion, selectivity, and yield were determined by the following calculation equations. For the conversion (olefin conversion (%)), selectivity ((poly)alkylene glycol monoalkyl ether selectivity (%)) and yield ((poly)alkylene glycol monoalkyl ether yield (%)), the values of the phases were calculated, and the total amounts thereof were used.

Conditions for the analysis by gas chromatography (GC) were as follows:

### <<GC Conditions>>

Gas chromatography instrument: trade name: Nexis™ GC-2030 available from Shimadzu Corporation
Column: trade name: DB-1 available from Agilent Technologies, Inc. (column length: 60 m, internal diameter: 0.25 mm, film thickness: 0.25 μm)
Detector: FID detection method
Injection volume: 1 μl
Linear velocity: 14 cm/see
Carrier gas: N₂

Temperature rising condition: temperature rising from 60 °C to 100 °C at 3 °C/min, then temperature rising to 320 °C at 10 °C/min, and holding at 320 °C for 30 minutes

OLEFIN CONVERSION (%)=(AMOUNT OF CONSUMED OLEFIN)/(AMOUNT OF CHARGED OLEFIN) (POLY) ALKYLENE GLYCOL MONOALKYL ETHER SELECTIVITY (%)=(MASS OF (POLY) ALKYLENE GLYCOL MONOALKYL ETHER PRODUCT)/(MASS OF CONSUMED OLEFIN) (POLY) ALKYLENE GLYCOL MONOALKYL ETHER YIELD (%)=(OLEFIN CONVERSION)×((POLY)ALKYLENE GLYCOL MONOALKYL ETHER SELECTIVITY) [Math. 2]

### Example 1

A 500-ml glass reactor equipped with a stirring blade and a reflux condenser was charged with 40 g (0.24 mol) of 1-dodecene, 59 g (0.95 mol) of monoethylene glycol, 105.5 g of diethylene glycol dimethyl ether, and 39.3 g of the catalyst (1). The gas phase part was replaced with nitrogen, and then held in a nitrogen atmosphere at normal pressure. Next, the temperature was raised to 135 °C while stirring at a rotational speed of 650 rpm, and the reaction was carried out at the same temperature for 4 hours. The liquid phase during the reaction was uniform. After the reaction, the reaction solution was cooled to 25 °C and separated into two phases. Obtained were 123.6 g of an upper phase (A) and 119.3 g of a lower phase (B). The upper phase (A) and the lower phase (B) were analyzed by gas chromatography, and the conversion of 1-dodecene and the selectivity and yield of ethylene glycol monododecyl ether are shown in Table 1. Next, the separated lower phase (B) was filtered to remove the catalyst, and then 13.4 g of diethylene glycol dimethyl ether as a solvent was distilled off by distillation (pressure: 4 kPa, column top temperature: from 80 to 100 °C). Thereafter, the reaction solution was cooled to 25 °C and separated into two phases, and 19.8 g of an upper phase (C) and 46.8 g of a lower phase (D) were obtained. The upper phase (A) and the upper phase (C) were distilled at 1 kPa to distill off the solvent and raw material components at a column top temperature of lower than 140 °C, and 18.9 g of ethylene glycol monododecyl ether was obtained at a column top temperature of from 140 to 143 °C.

### Example 2

The reaction was carried out in the same manner as in Example 1, except that the amount of diethylene glycol dimethyl ether was changed from 105.5 g in Example 1 to 193.9 g. The liquid phase during the reaction was uniform. After the reaction, the reaction solution was cooled to 25 °C, but no phase separation of the liquid phase was observed. 90.1 g of diethylene glycol dimethyl ether as the solvent was distilled off from the reaction solution by distillation (pressure: 4 kPa, column top temperature: from 80 to 100 °C). Then, the reaction solution was cooled to 25 °C and separated into two phases. Obtained were 122.2 g of an upper phase (A) and 118.0 g of a lower phase (B). The upper phase (A) and the lower phase (B) were analyzed by gas chromatography, and the conversion of 1-dodecene and the selectivity and yield of ethylene glycol monododecyl ether are shown in Table 1. Next, the separated lower phase (B) was filtered to remove the catalyst, and then 13.2 g of diethylene glycol dimethyl ether as a solvent was distilled off by distillation (pressure: 4 kPa, column top temperature: from 80 to 100 °C). Thereafter, the reaction solution was cooled to 25 °C and separated into two phases, and 19.6 g of an upper phase (C) and 46.3 g of a lower phase (D) were obtained. The upper phase (A) and the upper phase (C) were distilled at 1 kPa to distill off the solvent and raw material components at a column top temperature of lower than 140 °C, and 18.7 g of ethylene glycol monododecyl ether was obtained at a column top temperature of from 140 to 143 °C.

### Comparative Example 1

The reaction was carried out in the same manner as in Example 1, except that the amount of diethylene glycol dimethyl ether was changed from 105.5 g in Example 1 to 24.8 g. The solution was separated into two phases during the reaction, and was also separated into two phases when cooled to 25 °C. Obtained were 63.3 g of an upper phase (A) and 98.4 g of a lower phase (B). The upper phase (A) and the lower phase (B) were analyzed by gas chromatography, and the conversion of 1-dodecene and the selectivity and yield of ethylene glycol monododecyl ether are shown in Table 1. The upper phase (A) was distilled at 1 kPa to distill off the solvent and raw material components at a column top temperature of lower than 140 °C, and 12.3 g of ethylene glycol monododecyl ether was obtained at a column top temperature of from 140 to 143 °C.

### Example 3

A 500-ml glass reactor equipped with a stirring blade and a reflux condenser was charged with 40 g (0.20 mol) of 1-tetradecene, 50.6 g (0.82 mol) of monoethylene glycol, 105.0 g of diethylene glycol dimethyl ether, and 37.6 g of the catalyst (1). The gas phase part was replaced with nitrogen, and then held in a nitrogen atmosphere at normal pressure. Next, the temperature was raised to 135 °C while stirring at a rotational speed of 650 rpm, and the reaction was carried out at the same temperature for 4 hours. The liquid phase during the reaction was uniform. After the reaction, the reaction solution was cooled to 25 °C and separated into two phases. Obtained were 106.5 g of an upper phase (A) and 125.8 g of a lower phase (B). The upper phase (A) and the lower phase (B) were analyzed by gas chromatography, and the conversion of 1-tetradecene and the selectivity and yield of ethylene glycol monotetradecyl ether are shown in Table 1. Next, the separated lower phase (B) was filtered to remove the catalyst, and then 22.6 g of diethylene glycol dimethyl ether as a solvent was distilled off by distillation (pressure: 4 kPa, column top temperature: from 80 to 100 °C). Thereafter, the reaction solution was cooled to 25 °C and separated into two phases, and 18.7 g of an upper phase (C) and 46.9 g of a lower phase (D) were obtained. The upper phase (A) and the upper phase (C) were distilled at 0.5 kPa to distill off the solvent and raw material components at a column top temperature of lower than 142 °C, and 15.6 g of ethylene glycol monotetradecyl ether was obtained at a column top temperature of from 142 to 145 °C.

### Example 4

The reaction was carried out in the same manner as in Example 1, except that the amount of the catalyst (1) was changed from 39.3 g in Example 1 to 6.6 g. The liquid phase during the reaction was uniform. After the reaction, the reaction solution was cooled to 25 °C and separated into two phases. Obtained were 139.1 g of an upper phase (A) and 70.6 g of a lower phase (B). The upper phase (A) and the lower phase (B) were analyzed by gas chromatography, and the conversion of 1-dodecene and the selectivity and yield of ethylene glycol monododecyl ether are shown in Table 1. Next, the separated lower phase (B) was filtered to remove the catalyst, and then 9.1 g of diethylene glycol dimethyl ether as a solvent was distilled off by distillation (pressure: 4 kPa, column top temperature: from 80 to 100 °C). Thereafter, the reaction solution was cooled to 25 °C and separated into two phases, and 14.5 g of an upper phase (C) and 40.5 g of a lower phase (D) were obtained. The upper phase (A) and the upper phase (C) were distilled at 1 kPa to distill off the solvent and raw material components at a column top temperature of lower than 140 °C, and 17.2 g of ethylene glycol monododecyl ether was obtained at a column top temperature of from 140 to 143 °C.

### Example 5

The reaction was carried out in the same manner as in Example 3, except that the amount of the catalyst (1) was changed from 37.6 g in Example 3 to 5.6 g. The liquid phase during the reaction was uniform. After the reaction, the reaction solution was cooled to 25 °C and separated into two phases. Obtained were 128.4 g of an upper phase (A) and 70.8 g of a lower phase (B). The upper phase (A) and the lower phase (B) were analyzed by gas chromatography, and the conversion of 1-tetradecene and the selectivity and yield of ethylene glycol monotetradecyl ether are shown in Table 1. Next, the separated lower phase (B) was filtered to remove the catalyst, and then 11.7 g of diethylene glycol dimethyl ether as a solvent was distilled off by distillation (pressure: 4 kPa, column top temperature: from 80 to 100 °C). Thereafter, the reaction solution was cooled to 25 °C and separated into two phases, and 13.6 g of an upper phase (C) and 39.9 g of a lower phase (D) were obtained. The upper phase (A) and the upper phase (C) were distilled at 0.5 kPa to distill off the solvent and raw material components at a column top temperature of lower than 142 °C, and 14.5 g of ethylene glycol monotetradecyl ether was obtained at a column top temperature of from 142 to 145 °C.

### Example 6

The reaction was carried out in the same manner as in Example 1, except that the amount of diethylene glycol dimethyl ether was changed from 105.5 g in Example 1 to 66 g. The reaction solution was separated into two phases at the start of the reaction, but the liquid phase during the reaction (liquid phase after 120 minutes from the time when the temperature was raised to 135 °C) was uniform. After the reaction, the reaction solution was cooled to 25 °C and separated into two phases. Obtained were 97.0 g of an upper phase (A) and 106.3 g of a lower phase (B). The upper phase (A) and the lower phase (B) were analyzed by gas chromatography, and the conversion of 1-dodecene and the selectivity and yield of ethylene glycol monododecyl ether are shown in Table 1. The upper phase (A) was distilled at 1 kPa to distill off the solvent and raw material components at a column top temperature of lower than 140 °C, and 18.6 g of ethylene glycol monododecyl ether was obtained at a column top temperature of from 140 to 143 °C.

TABLE 1

| | | Ethylene glycol | Ethylene glycol |
|---|---|---|---|
| | Olefin | monoalkyl ether | monoalkyl ether |
| | conversion | selectivity | yield |
| | [%] | [%] | [%] |
| Example 1 | 47.6 | 84.8 | 40.4 |
| Example 2 | 46.5 | 88.5 | 41.2 |
| Comparative | 40.1 | 66.1 | 26.5 |
| Example 1 | | | |
| Example 3 | 42.5 | 83.6 | 35.6 |
| Example 4 | 39.5 | 91.7 | 36.2 |
| Example 5 | 35.5 | 89.8 | 31.9 |
| Example 6 | 50.2 | 82.8 | 41.6 |

### INDUSTRIAL APPLICABILITY

The (poly)alkylene glycol monoalkyl ether obtained by the present invention is useful as a raw material for a surfactant. A higher secondary alcohol alkoxylate adduct obtained by further adding an alkylene oxide is useful for a detergent, an emulsifier, a fiber treating agent, etc.

This application is based on JP 2022-169503 A filed on Oct. 21, 2022, the disclosure of which is incorporated herein by reference in its entirety.

1. A method for producing a (poly)alkylene glycol monoalkyl ether, the method comprising:
reacting an olefin with a (poly)alkylene glycol in the presence of a solvent, as a solution having a uniform liquid phase, at 110 to 250 °C using a crystalline metallosilicate as a catalyst;
separating a reaction solution obtained in the reaction into an upper phase (A) and a lower phase (B) at a temperature lower than 110 °C; and
obtaining a (poly)alkylene glycol monoalkyl ether from the upper phase (A) separated in the separation by distillation.

2. The production method according to claim 1,
further comprising removing, by distillation, a solvent component from the lower phase (B) separated in the separation, separating the lower phase (B) into an upper phase (C) and a lower phase (D), and obtaining the (poly)alkylene glycol monoalkyl ether from the separated upper phase (C) by distillation.

3. The production method according to claim 1, wherein the olefin is at least one selected from the group consisting of acyclic olefins having from 6 to 30 carbon atoms.

4. The production method according to claim 1, wherein the crystalline metallosilicate is a pentasil-type metallosilicate or a BEA-type metallosilicate.

5. The production method according to claim 1, wherein the solvent used in the reaction is a compound having a solubility parameter in a range of from 16 to 20 and represented by Formula (I):
R₁O—(AO)ₙ—R₂ (I)
where R₁ is a linear or branched alkyl group having from 1 to 4 carbon atoms, R₂ is a linear or branched alkyl group having from 1 to 4 carbon atoms or an acetyl group, A is a linear or branched alkylene group having from 2 to 4 carbon atoms, and n is from 1 to 4.

6. A method for producing a higher secondary alcohol alkoxylate adduct, the method comprising further adding an alkylene oxide to the (poly)alkylene glycol monoalkyl ether produced according to claim 1.
